(19) United States
(12) Patent Application Publication
(10) Pub. No.: US 2003/0216423 A1
(43) Pub. Date: Nov. 20, 2003

(54) Stable liquid and solid formulations

(76) Inventors: Sergio Ulloa, Delegacion Tlalpan (MX); Ramos Villacampa, Delegacion Benito Juarez (MX)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(21) Appl. No.: 10/276,637

(22) PCT Filed: May 22, 2001
(86) PCT No.: PCT/US01/16570

(30) Foreign Application Priority Data
May 24, 2000 (MX) 005129

(51) Int. Cl. A61K 31/473; A61K 31/137

(52) U.S. Cl. 514/290; 514/649

(57) ABSTRACT

Stable pharmaceutical compositions containing non-sedating antihistamines such as loratadine and a nasal decongestant such as pseudoephedrine sulfate and at least one pharmaceutically acceptable carrier, and stable pharmaceutical compositions containing non-sedating antihistamines such as loratadine, nasal decongestants such as pseudoephedrine, and an expectorant such as Ambroxol and at least one pharmaceutically acceptable carrier which are stable to chemical and physical degradation and microbial contamination are disclosed.

### BACKGROUND OF THE INVENTION

The present invention relates to stable liquid and solid pharmaceutical compositions containing loratadine and a nasal decongestant and optionally containing an expectorant and their use in the treatment of allergic and inflammatory conditions with cough and/or nasal congestion.

Loratadine is a long-acting, non-sedating antihistamine with selective peripheral histamines H1 reception approved for treatment of the symptoms of allergic reaction, for example, for the relief of nasal and non-nasal symptoms associated with seasonal allergic rhinitis or for treatment of chronic idopathic urticaria in human patients.

Nasal decongestants such as phenylpropanolamine, pseudoephedrine and their pharmaceutically acceptable salt such as the hydrogen chloride or hydrogen sulfate acid addition salts are useful for treating symptoms associated with allergic conditions such as seasonal allergic rhinitis or perennial allergic rhinitis as well as symptoms associated with the common cold including nasal congestion.

Mucolytic expectorants have been known in the art for some time. See Ambroxol, disclosed in U.S. Pat. No. 3,536,712.

Although useful, neither the non-sedating antihistamines nor the nasal decongestants nor the expectorants, in and of themselves, are capable of effectively treating the multitude of symptoms that may be associated with diseases of the respiratory tract, such as bronchitis and bronchial spasm, seasonal allergic rhinitis, perennial allergic rhinitis, common colds, sinusitis and concomitant symptoms associated with allergic asthma. The symptoms of such diseases may include sneezing, itching runny nose, nasal congestion, redness of the eye, tearing, itching of the ears or palate, and productive and non-productive coughs. It would be highly desirable to provide a formulation of these known separate drugs which enhances their individual efficacy and improves their overall efficacy.

Syrup formulations are commonly used for delivery of drugs particularly where the drugs are to be delivered to pediatric patients.

Loratadine syrup formulations have been found to be chemically and physically unstable and to have a proliferation of microbial agents such as molds, yeasts and bacteria.

It would be desirable to provide liquid and solid pharmaceutical compositions containing loratadine and a nasal decongestant and optionally containing an expectorant which are chemically and physically stable and resistant to microbial contamination. It would also be desirable to provide liquid pharmaceutical compositions which contain loratadine and a nasal decongestant and optionally contain an expectorant which are free of sugar, for example, glucose or sucrose, and ethanol and thus suitable for pediatric use.

### SUMMARY OF THE INVENTION

The present invention provides liquid pharmaceutical composition which are chemically and physically stable and protected from microbial contamination after storage at room temperature for up to 36 months. The liquid pharmaceutical compositions of the present invention are suitable for pediatric use.

The present invention provides a stable pharmaceutical composition comprising an effective amount of loratadine or a pharmaceutically acceptable salt thereof and an effective amount of nasal decongestant or a pharmaceutically acceptable salt thereof and optionally an effective amount of an expectorant or a pharmaceutically acceptable salt thereof and at least one pharmaceutically acceptable carrier.

In a preferred embodiment, the present invention provides a stable liquid pharmaceutical composition comprising an effective amount of loratadine or a pharmaceutically acceptable salt thereof and an effective amount of nasal decongestant or a pharmaceutically acceptable salt thereof and at least one pharmaceutically acceptable liquid carrier.

In another preferred embodiment, the present invention provides the present invention also provides a stable liquid pharmaceutical composition comprising an effective amount of loratadine or a pharmaceutically acceptable salt thereof and an effective amount of pseudoephedrine or a pharmaceutically acceptable salt thereof and at least one pharmaceutically acceptable liquid carrier.

In another preferred embodiment, the present invention provides a stable liquid pharmaceutical composition comprising an effective amount of loratadine or a pharmaceutically acceptable salt thereof an effective amount of pseudoephedrine or phenylpropanolamine or a pharmaceutically acceptable salt thereof, at least one pharmaceutically acceptable liquid carrier, and a combination of (1) a sweetener, (2) at least one pharmaceutically acceptable solvent, and (3) an amount of a buffer system to sufficient maintain the pH in the range of about 3.0 to about 5.0.

The present invention provides a stable liquid pharmaceutical composition comprising:

| Ingredient | mg/ml | |
|---|---|---|
| Loratadine | 0.50 | |
| Pseudoephedrine Sulfate | 6.00 | |
| Glycerin | 200.00 | |
| Propylene Glycol | 350.00 | |
| Sorbitol 70% Solution | 225.00 | |
| Sodium Saccharin | 0.40 | |
| Peach Flavor No. 609 | 2.50 | |
| Citric Acid, Anhydrous | 0.64 | |
| Sodium Citrate | 0.02 | |
| Purified Water | q.s. | |
| To make | 1.00 | ml |

The present invention provides a stable liquid pharmaceutical composition comprising:

| Ingredient | mg/ml | |
|---|---|---|
| Loratadine | 1.0 | |
| Pseudoephedrine Sulfate | 6.00 | |
| Glycerin | 200.00 | |
| Propylene Glycol | 350.00 | |
| Sorbitol 70% Solution | 225.00 | |
| Sodium Saccharin | 0.40 | |
| Peach Flavor No. 609 | 2.50 | |
| Citric Acid, Anhydrous | 0.64 | |
| Sodium Citrate | 0.02 | |
| Purified Water | q.s. | |
| To make | 1.00 | ml |

The stable liquid pharmaceutical compositions of the present invention such as the above-listed liquid pharmaceutical composition are useful for relief of the symptoms associated with upper airway passage congestion associated with disorders such as seasonal and perennial allergic rhinitis and for relief of the symptoms associated with upper respiratory infections such as the common cold, including nasal congestion.

In another preferred embodiment, the present invention provides a stable pharmaceutical composition comprising an effective amount of loratadine or a pharmaceutically acceptable salt thereof and an effective amount of nasal decongestant or a pharmaceutically acceptable salt thereof and an effective amount of an expectorant or a pharmaceutically acceptable salt thereof and at least one pharmaceutically acceptable carrier.

In another preferred embodiment, the present invention provides a stable oral tablet pharmaceutical composition comprising:

| | Concentration | | | |
|---|---|---|---|---|
| Name of Ingredients | (mg/tablet) | | Rationale | % Variation |
| Loratadine | 5.00 | | Active | |
| Ambroxol Hydrochloride | 30.00 | | Active | |
| Pseudoephedrine Sulfate | 60.00 | | Active | |
| Lactose Anhydrous | 84.75 | | Filler | ±20 |
| Corn Starch | 12.00 | | Disintegrant | ±20 |
| Cellulose Microcrystalline | 16.75 | | Disintegrant | ±10 |
| Colloidall Silicon Dioxide | 0.75 | | Glidant | ±10 |
| Magnesium Stearate | 0.75 | | Lubricant | ±10 |
| Tablet Weight | 210.00 | mg | | |

The above-listed oral tablets are suitable for twice-a-day administration.

In another preferred embodiment, the present invention provides a stable oral liquid pharmaceutical composition comprising:

| | Concentration | | |
|---|---|---|---|
| Name of Ingredients | mg/ml | Rationale | % Variation |
| Loratadine | 1.00 | Active | |
| Ambroxol Hydrochloride | 6.00 | Active | |
| Pseudoepherine Sulfate | 12.00 | Active | |
| Citric Acid | 0.40 | Buffer | ±10 |
| Glycerin | 150.00 | Solvent | ±20 |
| Propylene Glycol | 200.00 | Solvent | ±20 |
| Saccharin Sodium | 0.40 | Sweetener | ±10 |
| Sorbitol solution 70% | 450.00 | Solvent | ±20 |
| Flavor | 2.50 | Flavor | ±10 |

| | | | |
|---|---|---|---|
| Purified Water | q.s. | Solvent | ±20 |

| | | |
|---|---|---|
| To make | 1.00 | ml |

The preferred flavor is Peach Flavor No. 609, but other pharmaceutically acceptable flavors may also be used. The above-listed oral solution is suitable for twice-a-day administration.

In another preferred embodiment, the present invention provides a stable oral tablet pharmaceutical composition comprising:

| | Concentration | | | |
|---|---|---|---|---|
| Name of Ingredients | (mg/tablet) | | Rationale | % Variation |
| Loratadine | 3.33 | | Active | |
| Ambroxol Hydrochloride | 30.00 | | Active | |
| Pseudoephedrine Sulfate | 60.00 | | Active | |
| Lactose Anhydrous | 76.42 | | Filler | ±20 |
| Corn Starch | 12.00 | | Disintegrant | ±20 |
| Cellulose Microcrystalline | 16.75 | | Disintegrant | ±10 |
| Colloidall Silicon Dioxide | 0.75 | | Glidant | ±10 |
| Magnesium Stearate | 0.75 | | Lubricant | ±10 |
| Tablet Weight | 200.00 | mg | | |

The above-listed oral tablets are suitable for administration three-times-a-day.

In another preferred embodiment, the present invention provides a stable oral liquid pharmaceutical composition comprising:

| | Concentration | | |
|---|---|---|---|
| Name of Ingredients | mg/ml | Rationale | % Variation |
| Loratadine | 0.66 | Active | |
| Ambroxol Hydrochloride | 6.00 | Active | |
| Pseudoepherine Sulfate | 8.00 | Active | |
| Citric Acid | 0.40 | Buffer | ±10 |
| Glycerin | 150.00 | Solvent | ±20 |
| Propylene Glycol | 200.00 | Solvent | ±20 |
| Saccharin Sodium | 0.40 | Sweetener | ±10 |
| Sorbitol solution 70% | 448.00 | Solvent | ±20 |
| Flavor | 2.50 | Flavor | ±10 |

| | | | |
|---|---|---|---|
| Purified Water | q.s. | Solvent | ±20 |

| | | |
|---|---|---|
| To make | 1.00 | ml |

The above-listed solution is suitable for administration three-times-a-day.

The preferred flavor is Peach Flavor No. 609, but other pharmaceutically acceptable flavors may also be used.

Thus, the present invention provides a novel three-way combination of a slow release non-sedating antihistamine such as loratadine, and an expectorant and mucolytic agent such as ambroxol, and a nasal decongestant such as pseudoephedrine in a new liquid and tablet pharmaceutical preparations. These novel three-way combinations are indicated primarily for the symptomatic relief of allergic respiratory conditions associated with non-productive cough, nasal congestion and in the presence of mucus in the respiratory tract. These three-way pharmaceutical compositions of the present invention are also indicated in the treatment of those patients who show broncho-pulmonary conditions of allergic origin or other ethiology associated with cough, where viscosity and mucous adherence are increased, obstructing permeability of the airways. The principal indications include but are not limited to allergic rhinitis associated with cough and nasal congestion as well as acute, chronic, spasmodic and asthmatic bronchitis; bronchial asthma; bronchiectasis; sinusitis; otitis media; pneumonia; broncho-pneumonia; atelectasis by mucous obstruction; tracheotomy and as a pre and post prophylactic agent, especially in elderly patints where an allergic condition is suspected.

The present invention provides the use of loratadine and a nasal decongestant for the preparation of a liquid pharmaceutical composition for treatment of the symptoms associated with allergic reactions in a human.

The present invention provides the use of loratadine and a nasal decongestant for the preparation of a liquid pharmaceutical composition for treatment of the symptoms associated with allergic rhinitis and the common cold in a human.

The present invention provides the use of loratadine and a decongestant for the preparation of a liquid pharmaceutical composition for treatment of the symptoms associated with allergic rhinitis and the common cold including nasal congestion, sneezing, rhinorrhea, pruritus and lacrimation in a human.

The present invention provides the use of a non-sedating antihistamine in combination with an an expectorant and a nasal decongestant for the preparation of a medicament for the treatment of allergic and inflammatory respiratory conditions with cough, nasal congestion and the presence of mucus in the respiratory tract which comprises an effective amount of a non-sedating antihistamine in combination with an effective amount of an expectorant, and an effective amount of an nasal decongestant.

The nasal decongestant is preferably pseudoephedrine or a pharmaceutically acceptable salt thereof.

The two- and three-way liquid pharmaceutical composition of the present invention are stable to microbial contamination for periods of at least 4 months, preferably up to 36 months storage at room temperature (25° C.).

The two- and three-way liquid pharmaceutical composition of the present invention are stable to physical and chemical degradation of the active ingredients for periods of at least 4 months, preferably up to 36 months storage at room temperature (25° C.).

The two- and three-way liquid pharmaceutical composition of the present invention are stable to microbial contamination and to physical and chemical degradation of the active ingredients for periods of at least 4 months, preferably up to 36 months storage at room temperature (25° C.).

In a preferred embodiment, the liquid pharmaceutical compositions of the present invention are substantially free of sugar, for example, glucose or sucrose, and of ethanol and thus are suitable for pediatric use.

### DETAILED DESCRIPTION OF THE INVENTION

We have discovered liquid pharmaceutical compositions containing loratadine and a nasal decongestant, preferably pseudoephedrine, more preferably pseudoephedrine sulfate, as the active ingredients, which are stable to microbial contamination, including but not limited to bacterial contamination, and to physical and chemical degradation of the active ingredients for periods of at least 4 months, preferably up to 36 months storage at room temperature (25° C.). As shown in Table I, the preferred liquid pharmaceutical composition of Example 1 maintained a stable pH in the range of 3.3 to 4.3 and had no more than about 3% by weight change in the original amounts of loratadine and pseudoephedrine sulfate after storage at room temperature for at least 4 months and up to 36 months. Similar behavior is expected for the liquid and solid pharmaceutical compositions containing loratadine, a nasal decongestant such as pseudoephedrine, and a mucolytic agent such as ambroxol.

In addition, there was no change in the physical appearance and color and of the liquid pharmaceutical compositions of the present invention. The Antimocrobial Preservative Effectiveness (“APE”) Test performed in the liquid pharmaceutical compositions of the present invention such as Example 1 (See Table) demonstrated that the composition was free of microbial contamination after storage at room temperature for at least 4 months and up to 36 months.

The nasal decongestants useful in the present invention include the nasal decongestant pseudoephedrine and phenylpropanolamine and phaemaceutically acceptable salt thereof, especially the hydrogen chloride and hydrogen sulfate acid addition salts. Use of pseudoephedrine sulfate is preferred.

Typically suitable sweeteners include sugar, e.g. glucose, as well as artificial sweeteners such as saccharin, e.g. sodium saccharin, sorbitol, aspartame, sodium cyclamate and mixtures thereof. Preferably a combination of two artifical sweeteners is used; more preferably a combination of sodium saccharin and sorbitol is used. The sorbitol is normally a 70 weight % aqueous solution of sorbitol in purified water.

In preferred embodiments, the liquid pharmaceutical compositions of the present invention are substantially free of sugar, for example, glucose or sucrose, and ethanol and thus are suitable for pediatric use

The term “substantially free of sugar and ethanol” as used herein means that the liquid pharmaceutical compositions of the present invention contain less than 1%, more preferably less than 0.5%, and most preferably contain less than 0.1% by weight, of sugar, for example, glucose or sucrose, and of ethanol and thus are suitable for pediatric use.

Typically suitable flavoring agents include those flavoring agents approved for use in sweetened pharmaceuticals, foods, candies, and beverages. These flavoring agents impart flavors such as grape, cherry, citrus, peach, strawberry, bubble gum, peppermint and many others.

Typically suitable pharmaceutically acceptable solvents include alcohols and glycols, especially propylene glycol, ethanol and glycerin. The liquid pharmaceutical compositions indicated for pediatric use should be substantially free of, and most preferably should not contain ethanol. Use of a combination of propylene glycol and glycerin is preferred.

Typically suitable buffer systems include those capable of maintaining a pH in the range of 2.5 to 5.0, preferably about 2.9 to 4.3, more preferably 3.3-4.3. Preferred buffer systems include citric acid/sodium citrate; and acetic acid/sodium acetate. Use of sodium citrate/citric acid as the buffer system is preferred.

Normally, the pharmaceutically acceptable liquid carrier is purified water.

While we do not wish to be bound by any therapy, we believe that the unique combination of glycerin, propylene glycol, sorbitol and a sodium citrate/citric acid buffer system provide chemical and physical stability for the liquid pharmaceutical compositions of the present invention and provides the liquid pharmaceutical compositions of the present invention with effective antimicrobial protection from microbial contamination for periods of at least 4 months and up to 36 months when they are stored at room temperature. (See Table I) This particularly surprising and unexpected result is achieved without use of standard preservative such as sodium benzoate.

The present invention provides novel pharmaceutical formulations, which contain a combination of a non-sedating antihistamine and an expectorant or contain a combination of a non-sedating antihistamine, an expectorant, and a nasal decongestant. The formulations of the invention are useful in the prevention and/or treatment of allergic and inflammatory conditions of the skin or airway passages with cough. The formulations are useful for a wide range of patient ages; specific embodiments provided herein include a tablet form for administration to humans 12 years and older, and a pediatric solution for humans aged 6-12 years.

Also provided by the invention are methods for treating and/or preventing allergic and inflammatory conditions with related cough in humans in need of such treating and/or preventing which comprise administering an effective amount of a non-sedating antihistamine in combination with an expectorant.

The phrase “allergic and inflammatory conditions of the skin or airway passages” as used herein means those allergic and inflammatory conditions and symptoms found on the skin and in the upper and lower airway passages from the nose to the lungs. Typical allergic and inflammatory conditions of the skin or upper and lower airway passages include seasonal and perennial allergic rhinitis, allergic rhinitis associated with cough; non-allergic rhinitis, asthma including allergic and non-allergic asthma, sinusitis, colds, bronchopulmonary conditions of allergic origin associated with cough, where viscosity and mucous adherence are increased, obstructing permeability of the airways acute, chronic, spasmodic and asthmatic bronchitis, bronchial asthma, bronchiectasis, sinusitis, otitis media, pneumonia; bronchopneumonia, atelectasis by mucous obstruction, and dermatitis, especially allergic and atopic dermatitis, and urticaria and symptomatic dermographism as well as retinopathy, and small vessel diseases, associated with diabetes mellitus.

The term “a human age 6-12 years” as used herein means a male or female pediatric subject of 6 years of age to 12 years of age. The term “a human of 12 years and older” as used herein means a male or female pediatric subject of greater than 12 years of age to less than 18 years of age and adults of 18 years of age and older.

Although preferred embodiments of the present invention comprise treatment with loratadine and Ambroxol (See Examples 1 and 2), other antihistamines and expectorants are equally applicable in the compositions and methods taught herein. Following are non-inclusive lists of representative antihistamines and expectorants which may be used in the present invention.

I. Antihistamines

Loratadine is a non-sedating antihistamine whose technical name is 11-(4-piperidylidene)-5H-benzo-[5,6]-cyclohepta-[1,2-b]-pyridine. The compound is described in U.S. Pat. No. 4,282,233. Loratadine is a potent tricyclic and antihistaminic drug with a selective antagonist of peripheric H₁ receptors activity. The amount of loratadine which can be employed in a unit (i.e., single) dosage form of the present compositions can range from about 1.0 mg to about 15.0 mg, also from about −2.5 mg to about 10.0 mg, preferably from about 5.0 mg. to about 10.0 mg.

Desloratadine is a non-sedating long acting histamine antagonist with potent selective peripheral H1-receptor antagonist activity. Following oral administration, loratadine is rapidly metabolized to descarboethoxyloratadine or desloratadine, a pharmacologically active metabolite. U.S. Pat. Nos. 4,659,716, 5,595,997 and 4,804,666 disclose methods of making desloratadine, pharmaceutical compositions containing it and methods for using it to treat various disease states in mammals. The amount of desloratadine which can be employed in a unit (i.e., single) dosage form of the present compositions can range from about 0.75 mg to about 7.5 mg, also from about 1.25 mg to about 5.0 mg, preferably from about 2.5 mg. to about 5.0 mg.

Descarboethoxyloratadine (DCL) is non-sedating antihistamine, whose technical name is 8-chloro-6,11-dihydro-11-(4-piperidylidene)-5H-benzo[5,6]cyclohepta[1,2]pyridine. This compound is described in Quercia, et al., Hosp. Formul., 28: 137-53 (1993), in U.S. Pat. No. 4,659,716, and in WO 96/20708. DCL is an antagonist of the H-1 histamine receptor protein. The H-1 receptors are those that mediate the response antagonized by conventional antihistamines. H-1 receptors are present, for example, in the ileum, the skin, and the bronchial smooth muscle of man and other mammals. The amount of DCL which can be employed in a unit (i.e. single) dosage form of the present compositions can range from about 2.5 to about 20 mg, also from about 5 to about 10 mg, preferably about 5 or 7.5 mg.

Fexofenadine (MDL 16,455A) is a non-sedating antihistamine, whose technical name is 4-[1-hydroxy-4-(4-hydroxy-diphenylmethyl)-1-piperidinyl)butyl]-α,α-dimethyl-benzene acetic acid. Preferably the pharmaceutically acceptable salt is the hydrochloride, also known as fexofenadine hydrochloride. The amount of fexofenadine which can be employed in a unit dosage form of the present composition can range from about 40 to 200 mg, also from about 60 to about 180 milligrams, also about 120 milligrams.

II. Expectorants

Ambroxol is a bromhexine metabolite, chemically identified as trans-4(2-amino-3,5-dibromobenzil, amine) ciclohexane hydrochloride, which has been widely used during more than two decades as an expectorant agent or stimulating pulmonary surfactant factor. The compound is described in U.S. Pat. No. 3,536,712. The amount of ambroxol which can be employed in a unit dosage form can range from about 30.0 to about 60.0 mg, preferably about 60.0 mg.

Guaiafenesin is an expectorant, whose technical name is 3-(2-methoxyphenoxy)-1,2-propanediol. The compound is described in U.S. Pat. No. 4,390,732. The amount of guaiafenesin which can be employed in a unit dosage form can range from about 300.0 to about 1200.0 mg, preferably about 1200.0 mg.

Terpin hydrate is an expectorant, whose technical name is 4-hydroxy-α,α,4-trimethylcyclohexane-methanol. The amount of terpin hydrate that employed in a unit dosage form of the present composition can range from 85.0 to 680.0 milligrams.

For preparing pharmaceutical compositions from the compounds described by this invention, inert, pharmaceutically acceptable diluents, excipients and carriers can be either solid or liquid. Solid form preparations include powders, tablets, dispersible granules, capsules (either solid-filled, semi-solid filled or liquid-filled), sachets and suppositories. The powders and tablets may be comprised of from about 5 to about 95 percent active ingredient. Suitable solid carriers are known in the art, e.g. magnesium carbonate, magnesium stearate, talc, sugar or lactose. Tablets, powders, sachets and capsules can be used as sold dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa. Moreover, when desired or needed, suitable binders, lubricants, disintegrating agents and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Among the lubricants that may be mentioned for use in these dosage forms are, metallic stearates, talc, starch powder, stearic acid, different grades of polyethylene-glycol and the like. Disintegrants include starch, methylcellulose, guar gum and the like. Sweetening and flavoring agents and preservatives may also be included where appropriate.

Additionally, the compositions of the present invention may be formulated in sustained release form to provide the rate controlled release of any one or more of the components or active ingredients to optimize the therapeutic effects. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Liquid form preparations include solutions, suspensions and emulsions. As an example may be mentioned water or water-propylene glycol solutions for parenteral injection or addition of sweeteners and pacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 0.01 mg to about 1000 mg preferably from about 0.01 mg to about 750 mg more preferably from about 0.01 mg to about 750 mg and most preferably from about 0.01 mg to about 250 mg according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention will be regulated according to the judgment of the attending clinician considering such factors as age, condition and size of the patient as well as severity of the symptoms being treated. A typical recommended daily dosage regimen for oral administration can range from about 5.0 to 10.0 mg/day of loratadine and 30 to 60.0 mg/day of ambroxol, in two to four divided doses.

Dosage form—non-sedating antihistamine and expectorant formulated into a delivery system, i.e., tablet, capsule, oral gel, powder for constitution or suspension in association with inactive ingredients.

Capsule—refers to a special container or enclosure made of methyl cellulose, polyvinyl alcohols, or denatured gelatins or starch for holding or containing compositions comprising non-sedating antihistamine and an expectorant. Hard shell capsules are typically made of blends of relatively high gel strength bone and pork skin gelatins. The capsule itself may contain small amounts of dyes, opaquing agents, plasticizers and preservatives.

Tablet—refers to a compressed or molded solid dosage form containing the active ingredients with suitable diluents. The tablet can be prepared by compression of mixtures or granulations obtained by wet granulation, dry granulation or by compaction.

Oral gels—refers to a non-sedating antihistamine and an expectorant dispersed or solubilized in a hydrophilic semi-solid matrix.

Powders for constitution—refers to powder blends containing the active ingredients and suitable diluents which can be suspended in water or juices.

Diluent—refers to substances that usually make up the major portion of the composition or dosage form. Suitable diluents include sugars such as lactose, sucrose, mannitol and sorbitol; starches derived from wheat, corn rice and potato; and celluloses such as microcrystalline cellulose. The amount of diluent in the composition can range from about 10 to about 90% by weight of the total composition, preferably from about 25 to about 75%, more preferably from about 30 to about 60% by weight, even more preferably from about 12 to about 60%.

Disintegrants—refers to materials added to the composition to help it break apart (disintegrate) and release the medicaments. Suitable disintegrants include starches; “cold water soluble” modified starches such as sodium carboxymethyl starch; natural and synthetic gums such as locust bean, karaya, guar, tragacanth and agar; cellulose derivatives such as methylcellulose and sodium carboxymethylcellulose; microcrystalline celluloses and cross-linked microcrystalline celluloses such as sodium croscarmellose; alginates such as alginic acid and sodium alginate; clays such as bentonites; and effervescent mixtures, cross-linked povidones. The amount of disintegrant in the composition can range from about 2 to about 15% by weight of the composition, more preferably from about 4 to about 10% by weight.

Binders—refers to substances that bind or “glue” powders together and make them cohesive by forming granules, thus serving as the “adhesive” in the formulation. Binders add cohesive strength already available in the diluent or bulking agent. Suitable binders include sugars such as sucrose; starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropylmethylcellulose; polyvinylpyrrolidone; and inorganics such as magnesium aluminum silicate. The amount of binder in the composition can range from about 2 to about 20% by weight of the composition, more preferably from about 3 to about 10% by weight, even more preferably from about 3 to about 6% by weight.

Lubricant—refers to a substance added to the dosage form to enable the tablet, granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene-glycols and d′l-leucine. Lubricants are usually added at the very last step before compression, since they must be present on the surfaces of the granules and in between them and the parts of the tablet press. The amount of lubricant in the composition can range from about 0.2 to about 5% by weight of the composition, preferably from about 0.5 to about 2%, more preferably from about 0.3 to about 1.5% by weight.

Glidants—materials that prevent caking and improve the flow characteristics of granulations, so that flow is smooth and uniform. Suitable glidants include silicon dioxide and talc. The amount of glidant in the composition can range from about 0.1% to about 5% by weight of the total composition, preferably from about 0.5 to about 2% by weight.

Coloring agents—excipients that provide coloration to the composition or the dosage form. Such excipients can include food grade dyes and food grade dyes adsorbed onto a suitable adsorbent such as clay or aluminum oxide. The amount of the coloring agent can vary from about 0.1 to about 5% by weight of the composition, preferably from about 0.1 to about 1%.

Bioavailability—refers to the rate and extent to which the active drug ingredient or therapeutic moiety is absorbed into the systemic circulation from an administered dosage form as compared to a standard or control.

Conventional methods for preparing tablets are known. Such methods include dry methods such as direct compression and compression of granulation produced by compaction, or wet methods or other special procedures.

The broad scope of this invention is best understood with reference to the following examples, which are not intended to limit the invention to specific embodiments.

The present invention is further described by means of the following examples which are not intended to limit the scope of the present invention as defined by the attached claims.

### EXAMPLE 1

A solution was formulated to contain the following ingredients:

| Ingredient | mg/ml | | gl1000 L | |
|---|---|---|---|---|
| Loratadine | 0.50 | | 500 | |
| Pseudoephedrine Sulfate | 6.00 | | 6,000 | |
| Glycerin | 200.00 | | 200,000 | |
| Propylene Glycol | 350.00 | | 350,000 | |
| Sorbitol 70% Solution | 225.00 | | 225,000 | |
| Sodium Sacchariin | 0.40 | | 400 | |
| Peach Flavor No. 609 | 2.50 | | 2,500 | |
| Citric Acid, Anhydrous | 0.64 | | 640 | |
| Sodium Citrate | 0.02 | | 20 | |
| Purified Water | q.s | | q.s. | |
| To make | 1.00 | ml | 1,000 | L |

A 1000L batch of the solution of Example 1 is prepared using the following procedure:

1. Into a suitable size, stainless steel, compounding tank equipped with a stainless steel agitator, charge the Propylene Glycol, slowly agitate and heat to 40° C. ±5° C.

2. Charge the Loratadine to the heated Propylene Glycol in step 1. Continue agitation until completely dissolved.

3. Maintain mixing and charge the Glycerin USP and Sorbitol Solution to the batch in step 2. Mix until the batch is homogenous.

4. Into a suitable size, stainless steel vessel, equipped with a stainless steel agitator, charge 40 liters of Purified water and start agitation. Charge the following ingredients to the Purified Water, Sodium Saccharin, Sodium Citrate, Citric Acid, and Pseudoephedrine Sulfate. Ensure that each ingredient is dissolved before beginning the addition of the next.

5. Add the active solution from Step 4 to the batch in Step 3, use Purified Water to rinse the active solution vessel several times: (2 to 3 liters: three times); add the rinses to the batch. Mix until the batch is homogeneous.

6. Maintain agitation, charge the Flavor Peach to the batch in step 5. Mix until homogenous.

7. Check the pH, and adjust it is necessary to a value of 3.5±0.1 using Citric Acid as a 10% solution in purified water. Record the volume of solution added and the resulting pH.

8. Bring the batch to volume (1,000 liters) with Purified Water and mix until homogenous.

9. Filter the batch through an Ertel Filter press (or equivalent) equipped with a suitable asbestos-free filter pad, circulating the batch back into the tank until the outgoing filtrate is sparkling clear. Then filter the batch into a suitable stainless steel holding tank.

10. Fill into approved containers.

Typically suitable containers for liquid compositions of the present invention include type III amber glass bottle and a high density polyethylene (“HDPE”) bottle with 30 mL graduated spoon. Store between 2° and 30° C.

### EXAMPLE 2

The procedure of Example 1 is used to prepare the following solution.

| Ingredient | mg/ml | |
|---|---|---|
| Loratadine | 1.0 | |
| Pseudoephedrine Sulfate | 6.00 | |
| Glycerin | 200.00 | |
| Propylene Glycol | 350.00 | |
| Sorbitol 70% Solution | 225.00 | |
| Sodium Saccharin | 0.40 | |
| Peach Flavor No. 609 | 2.50 | |
| Citric Acid, Anhydrous | 0.64 | |
| Sodium Citrate | 0.02 | |
| Purified Water | q.s | |
| To make | 1.00 | ml |

The solution formulation may also be stored in 30 mL type III amber glass bottle with dropper assembly tops. Use of 30 mL HDPE white opaque bottles is also possible. The solution formulation of Example 1 was stored in 30 mL type III amber glass bottles with tin plate cap with a vinyl on pulp cap liner at room temperature, 35° and 45° C. Samples were removed and tested for degradation by HPLC, using loratadine and pseudoephedrine standards. The pH, Alpha color and APE tests were also performed. The results are summarized in Table I. Similar results are expected for the solution formulation of Example 2.

The solution formulations of Examples 1 and 2 are indicated to be useful to treat pediatric patients of 2 to 8 years of age for the relief of symptoms associated with allergic rhinitis and the common cold including nasal congestion, sneezing, rhinorrhea, pruritus and lacrimation. In addition, the solution formulations of the present invention are useful when both the antihistamine properties of loratadine and the decongestant properties of pseudoephedrine sulfate are desired in any patient, including pediatric patients of 2-8 years of age and older. Thus, the stable liquid pharmaceutical compositions of the present invention are useful for relief of the symptoms associated with upper airway passage congestion associated with disorders such as seasonal and perennial allergic rhinitis and for relief of the symptoms associated with upper respiratory infections such as the common cold including nasal congestion, The precise dose and dosage regimen will be determined by the attending physician in view of the age and medical condition of the patient as well as the severity of the symptoms associated with upper airway passage congestion and the severity of symptoms associated with upper respiratory infections such as the common cold including nasal congestion.

The liquid pharmaceutical compositions of the present invention containing 0.50 mg/ml of loratadine and 6.0 mg/ml of pseudoephedrine sulfate may be administered to a pediatric human patient of 2 to 8 years of age in accordance with the following regimen:

| | | Volume (ml) of the liquid |
|---|---|---|
| Age Range (years) | Body Weight (Kg) | composition every 12 hr |
| 2 to 5 | 12.5 to 18.5 | 2.5 |
| 6 to 8 | >18.5 to <30 | 5.0 |

The liquid pharmaceutical compositions of the present invention containing 1.0 mg/ml of loratadine and 6.0 mg/ml of pseudoephedrine sulfate may be administered to a pediatric human patient of 2 to 8 years of age in accordance with the following regimen:

| | | Volume (ml) of the liquid |
|---|---|---|
| Age (years) | Body Weight (Kg) | composition every 12 hr |
| 2 | 12.5 | 2 |
| 3 | 14.5 | 2.5 |
| 4 | 16.5 | 3.0 |
| 5 | 18.5 | 3.5 |
| 6 | 20.5 | 4.0 |
| 7 | 23.6 | 4.5 |
| 8 | 26-<30 | 5.0 |

### EXAMPLES 3A-D

### Novel Pharmaceutical Compositions

The present invention provides a novel combination of a slow release non-sedating antihistamine such as loratadine, and an expectorant and mucolytic agent such as ambroxol, in a new pharmaceutical preparation. The combination is indicated primarily in the treatment of those patients who show bronchopulmonary conditions of allergic origin associated with cough, where viscosity and mucous adherence are increased, obstructing permeability of the airways. The principal indications include but are not limited to allergic rhinitis associated with cough; acute, chronic, spasmodic and asthmatic bronchitis; bronchial asthma; bronchiectasis; sinusitis; otitis media; pneumonia; bronchopneumonia; atelectasis by mucous obstruction; tracheotomy and as a pre and post prophylactic agent.

### EXAMPLE 3A

| | Concentration | | | |
|---|---|---|---|---|
| Name of Ingredients | (mg/tablet) | | Rationale | % Variation |
| Loratadine | 5.00 | | Active | |
| Ambroxol Hydrochloride | 30.00 | | Active | |
| Pseudoephedrine Sulfate | 60.00 | | Active | |
| Lactose Anhydrous | 84.75 | | Filler | ±20 |
| Corn Starch | 12.00 | | Disintegrant | ±20 |
| Cellulose Microcrystalline | 16.75 | | Disintegrant | ±10 |
| Colloidall Silicon Dioxide | 0.75 | | Glidant | ±10 |
| Magnesium Stearate | 0.75 | | Lubricant | ±10 |
| Tablet Weight | 210.00 | mg | | |

Manufacturing Procedure:

1. Charge into a suitable size, stainless shell, twin shell blender the Loratadine Micronized, Colloidal silicon dioxide, and approximately ½ the amount of the Lactose Anhydrous. Blend for 10 minutes. 2. Discharge the contents of the blender from step 1 and pass the blend through a mechanical sieve or Oscillator equipped with a 40 mesh screen and return the sample to the twin shell blender.

3. Pass the remaining quantity of Lactose Anhydrous through the same mechanical sieve or oscillator from step 2 and return the sample to the blender from step 2.

4. Charge into the blender from step 2, the Ambroxol Hydrochloride, Pseudoephedrine sulphate, Corn Starch, Cellulose microcrystalline. Blend for 20 minutes.

5. Charge into the blender from step 2, the Magnesium Stearate. Blend for 3 minutes.

6. Discharge the contents of the blender from step 5, into appropriate storage containers, lined with two polyethylene bags; tie the bags separately, and record the weight.

7. Using a suitable tablet press, compress the blend from step 6 into tablets.

### EXAMPLE 3B

| | Concentration | | |
|---|---|---|---|
| Name of Ingredients | (mg/ml) | Rationale | % Variation |
| Loratadine | 1.00 | Active | |
| Ambroxol Hydrochloride | 6.00 | Active | |
| Pseudoepherine Sulfate | 12.00 | Active | |
| Citric Acid | 0.40 | Buffer | ±10 |
| Glycerin | 150.00 | Solvent | ±20 |
| Propylene Glycol | 200.00 | Solvent | ±20 |
| Saccharin Sodium | 0.40 | Sweetener | ±10 |
| Sorbitol solution 70% | 450.00 | Solvent | ±20 |
| Flavor | 2.50 | Flavor | ±10 |

| | | | |
|---|---|---|---|
| Purified Water | q.s. | Solvent | ±20 |

| | | |
|---|---|---|
| To make | 1.00 | ml |

1. Peach Flavor No. 609

Manufacturing Procedure:

1. Into a suitable size, stainless steel, compounding tank, charge the Propylene Glycol; add and dissolve the following: Saccharin Sodium, Loratadine Micronized. Ensure that each ingredient is dissolved before the addition of the next.

2. Into a suitable size, jacketed stainless steel compounding tank equipped with a stainless steel agitator, charge a portion of the Purified Water and heat to 60° C.-65° C.

3. With continuous agitation, add and dissolve the Citric Acid. Mix thoroughly until a clear solution is obtained. Charge the solution to the batch and mix until homogeneous. Rinse the tank with two portions of Purified Water.

4. Cool the batch and maintain temperature at 40° C.-45° C.

5. With continued agitation, add and dissolve the Ambroxol Hydrochloride, the Pseudoephedrine sulfate. Mix thoroughly until a clear solution is obtained.

6. Charge the Glycerin to the batch and mix until homogeneous.

7. Charge the Sorbitol Solution to the batch and mix until homogeneous. Cool the batch to room temperature (20° C.-25° C.).

8. Charge the Flavor Artificial to the batch and mix until homogeneous.

9. Check and adjust the pH to 2.9±0.2 if necessary with 1% Citric Acid Solution or 0.1 N Sodium Hydroxide Solution.

10. Bring the batch to volume with Purified Water and mix until homogeneous.

11. Filter the batch through an Ertel Filter press (or equivalent) equipped with suitable asbestos-free filter pads; circulate the batch back into the tank until the outgoing filtrate is sparklingly clear. Then filter the batch into a suitable steel holding tank.

### EXAMPLE 3C

| | Concentration | | | |
|---|---|---|---|---|
| Name of Ingredients | (mg/tablet) | | Rationale | % Variation |
| Loratadine | 3.33 | | Active | |
| Ambroxol Hydrochloride | 30.00 | | Active | |
| Pseudoephedrine Sulfate | 60.00 | | Active | |
| Lactose Anhydrous | 76.42 | | Filler | ±20 |
| Corn Starch | 12.00 | | Disintegrant | ±20 |
| Cellulose Microcrystalline | 16.75 | | Disintegrant | ±10 |
| Colloidall Silicon Dioxide | 0.75 | | Glidant | ±10 |
| Magnesium Stearate | 0.75 | | Lubricant | ±10 |
| Tablet Weight | 200.00 | mg | | |

Manufacturing Procedure:

1. Charge into a suitable size, stainless steel, twin shell blender the Loratadine Micronized, Colloidal silicon dioxide, and approximately ½ the amount of the Lactose Anhydrous. Blend for 10 minutes.

2. Discharge the contents of the blender from step 1 and pass the blend through mechanical sieve or Oscillator equipped with a 40 mesh screen and return the material to the twin shell blender.

3. Pass the remaining quantity of Lactose Anhydrous through the same mechanical sieve or oscillator from step 2 and return the material to the blender from step 2.

4. Charge into the blender from step 2, the Ambroxol Hydrochloride, Pseudoephedrine sulphate, Corn Starch, Cellulose microcrystalline. Blend for 20 minutes.

5. Charge into the blender from step 2, the Magnesium Stearate. Blend for 3 minutes.

6. Discharge the contents of the blender from step 5, into appropriate storage containers, lined with two polyethylene bags. Tie the bags separately, and record the weight.

7. Using a suitable tablet press, compress the blend from step 6 into tablets.

### EXAMPLE 3D

| | Concentration | | |
|---|---|---|---|
| Name of Ingredients | (mg/ml) | Rationale | % Variation |
| Loratadine | 0.66 | Active | |
| Ambroxol Hydrochloride | 6.00 | Active | |
| Pseudoepherine Sulfate | 8.00 | Active | |
| Citric Acid | 0.40 | Buffer | ±10 |
| Glycerin | 150.00 | Solvent | ±20 |
| Propylene Glycol | 200.00 | Solvent | ±20 |
| Saccharin Sodium | 0.40 | Sweetener | ±10 |
| Sorbitol solution 70% | 448.00 | Solvent | ±20 |
| Flavor | 2.50 | Flavor | ±10 |

| | | | |
|---|---|---|---|
| Purified Water | q.s. | Solvent | ±20 |

| | | |
|---|---|---|
| To make | 1.00 | ml |

1. Peach Flavor No. 609

Manufacturing Procedure:

1. Into a suitable size, stainless steel, compounding tank, charge the Propylene Glycol, add and dissolve the following: Saccharin Sodium, Loratadine Micronized. Ensure that each ingredient is dissolved before the addition of the next.

2. Into a suitable size, jacketed stainless steel compounding tank equipped with a stainless steel agitator, charge a portion of the Purified Water and heat to 60° C.-65° C.

5. With continued agitation, add and dissolve the Ambroxol Hydrochloride, the Pseudoephedrine sulfate. Mix thoroughly until a clear solution is obtained.

6. Charge the Glycerin to the batch and mix until homogeneous.

7. Charge the Sorbitol Solution to the batch and mix until homogeneous. Cool the batch to room temperature (20° C.-25° C.).

8. Charge the Flavor Artificial to the batch and mix until homogeneous.

9. Check and adjust the pH to 2.9±0.2 if necessary with 1% Citric Acid Solution or 0.1 N Sodium Hydroxide Solution.

10. Briing the batch to volume with Purified Water and mix until homogeneous.

11. Filter the batch through an Ertel Filter press (or equivalent) equipped with suitable asbestos-free filter pads. Circulate the batch back into the tank until the outgoing filtrate is sparklingly clear. Then filter the batch into a suitable stainless steel holding tank.

### Dosage and Administration

For the Tablets of Examples 3A and 3C, adults and children 12 years of age and older may take two tablets a day of these prepared in accordance with Example 3A or three tablets a day of those prepared in accordance with Example 3C.

The solutions of Examples 3B and 3D may be administered as follows:

(1) Adults and children 12 years of age and older may take 5 ml (1 teaspoon) of the solution of Example 3B twice a day (BID) or 5 ml (1 teaspoon) of the solution of Example 3D three times a day (TID);

(2) Children 6 years of age to less than 12 years of age may take 2.5 ml (¼ teaspoon) of the solution of Example 3B twice-a-day or 2.5 ml (¼ teaspoon) of solution of Example 3D three-times-a-day; and

(3) Children 2 years of age to less than 6 years of age may take 1.25 ml (½ teaspoon) of the solution of Example 3B twice-a-day or 1.25 ml (½ teaspoon) of the solution of Example 3D three-times-a-day.

Of course, the precise dose and frequency of administration of the pharmaceutical compositions of the present invention may be adjusted by the attending physician in view of the patient's age, weight, severity of disease and other health problems.

### Clinical Studies

The safety and efficacy of each separate component of the loratadine/ambroxol combination and the loratadine/ambroxol/pseudoephedrine combination are well established. Studies of acute toxicity carried out with loratadine (in extensive clinical and preclinical programs) pseudoephedrine and ambroxol components have confirmed the low potential for systemic toxicity, which was expected for these combinations.

Other modifications well known to those skilled in the art may be made in the solution formulations of the present invention. Typical suitable additional drugs include analgesics such as aspirin, acetaminophen, ibuprofen, naproxen or ketoprofen for the relief of pain and, except in the case of acetaminophen, the relief of inflammation. Antitussives, such as codeine, hydrocodone or dextromethorphan, for relief from coughing, and expectorants such as guaifenesin, for increasing cough productivity, may also be included in the syrup formulations to form combination products. Any of these additional drugs including salts thereof, and other drugs from the same therapeutic classes may be added to the solution formulations without departing from the scope of the present invention.

1. A stable pharmaceutical composition comprising an effective amount of loratadine or a pharmaceutically acceptable salt thereof and an effective amount of nasal decongestant or a pharmaceutically acceptable salt thereof and optionally an effective amount of an expectorant or a pharmaceutically acceptable salt thereof and at least one pharmaceutically acceptable carrier.

2. A stable liquid pharmaceutical composition comprising an effective amount of loratadine or a pharmaceutically acceptable salt thereof and an effective amount of pseudoephedrine or a pharmaceutically acceptable salt thereof and at least one pharmaceutically acceptable liquid carrier.

3. A stable pharmaceutical composition comprising an effective amount of loratadine or a pharmaceutically acceptable salt thereof and an effective amount of nasal decongestant or a pharmaceutically acceptable salt thereof and an effective amount of an expectorant or a pharmaceutically acceptable salt thereof and at least one pharmaceutically acceptable carrier.

4. The pharmaceutical composition of any preceding claim wherein the nasal decongestant is pseudoephedrine or a pharmaceutically acceptable salt thereof.

5. The pharmaceutical composition of claim 1 or 3 wherein the stable pharmaceutical composition is a liquid dosage form and contains at least one pharmaceutically acceptable liquid carrier.

6. The pharmaceutical composition of claim 1 or 3 wherein the stable pharmaceutical composition is a solid dosage form.

7. The pharmaceutical composition of claim 2 or 5 which contains an antimicrobial effective amount of a sweetener, propylene glycol and glycerin.

8. The pharmaceutical composition of claims 1,2 or 3 which contains about 0.4 to about 0.7 mg/ml of loratadine.

9. A liquid pharmaceutical composition stable to microbial contamination comprising an effective amount of loratadine or a pharmaceutically acceptable salt thereof an effective amount of pseudoephedrine or phenylpropanolamine or a pharmaceutically acceptable salt thereof, and a combination of (1) a sweetener, (2) at least one pharmaceutically acceptable liquid carrier, and (3) an amount of a buffer system to sufficient maintain the pH in the range of about 3.0 to about 5.0.

10. A stable pharmaceutical composition stable to microbial contamination comprising an effective amount of loratadine or a pharmaceutically acceptable salt thereof an effective amount of pseudoephedrine or phenylpropanolamine or a pharmaceutically acceptable salt thereof, and an effective amount of an expectorant or a pharmaceutically acceptable salt thereof, at least one pharmaceutically acceptable carrier.

11. The pharmaceutical composition of claim 10 wherein the stable pharmaceutical composition is a liquid dosage form and contains a combination of (1) a sweetener, (2) at least one pharmaceutically acceptable liquid carrier, and (3) an amount of a buffer system to sufficient maintain the pH in the range of about 2.5 to about 5.0.

12. The pharmaceutical composition of claim 10 wherein the stable pharmaceutical composition is a solid dosage form.

13. The pharmaceutical composition of claim 9 or 10 wherein the sweetener is a combination of sodium saccharide and sorbitol.

14. A stable liquid pharmaceutical composition comprising:

| Ingredient | mg/ml | |
|---|---|---|
| Loratadine | 0.50 | |
| Pseudoephedrine Sulfate | 6.00 | |
| Glycerin | 200.00 | |
| Propylene Glycol | 350.00 | |
| Sorbitol 70% Solution | 225.00 | |
| Sodium Saccharin | 0.40 | |
| Peach Flavor No. 609 | 2.50 | |
| Citric Acid, Anhydrous | 0.64 | |
| Sodium Citrate | 0.02 | |
| Purified Water | q.s. | |
| To make | 1.00 | ml |

15. A stable liquid pharmaceutical composition comprising:

| Ingredient | mg/ml | |
|---|---|---|
| Loratadine | 1.0 | |
| Pseudoephedrine Sulfate | 6.00 | |
| Glycerin | 200.00 | |
| Propylene Glycol | 350.00 | |
| Sorbitol 70% Solution | 225.00 | |
| Sodium Saccharin | 0.40 | |
| Peach Flavor No. 609 | 2.50 | |
| Citric Acid, Anhydrous | 0.64 | |
| Sodium Citrate | 0.02 | |
| Purified Water | q.s. | |
| To make | 1.00 | ml |

16. The liquid pharmaceutical composition of any preceding claim that is stable to microbial contamination for periods of at least 4 months, preferably up to 36 months storage at room temperature.

17. The liquid pharmaceutical composition of any preceding claim that is stable to physical and chemical degradation of the active ingredients for periods of at least 4 months, preferably up to 36 months storage at room temperature.

18. The liquid pharmaceutical composition of any preceding claim that is stable to microbial contamination and to physical and chemical degradation of the active ingredients for periods of at least 4 months, preferably up to 36 months storage at room temperature.

19. The liquid pharmaceutical composition of any preceding claim that is substantially free of sugar such as glucose or sucrose and of ethanol and suitable for pediatric use.

20. The use of loratadine and a nasal decongestant for the preparation of a liquid pharmaceutical composition for treatment of the symptoms associated with allergic reactions which comprises effective amount of a non-sedating antihistamine in combination with an effective amount of an nasal decongestant.

21. The use of loratadine and a nasal decongestant for the preparation of a liquid pharmaceutical composition for treatment of the symptoms associated with allergic rhinitis and the common cold which comprises effective amount of a non-sedating antihistamine in combination with an effective amount of an nasal decongestant.

22. The use of loratadine and a nasal decongestant for the preparation of a liquid pharmaceutical composition for treatment of the symptoms associated with allergic rhinitis and the common cold including nasal congestion, sneezing, rhinorrhea, pruritus and lacrimation which comprises effective amount of a non-sedating antihistamine in combination with an effective amount of an nasal decongestant.

23. The use of a non-sedating antihistamine in combination with an expectorant and a nasal decongestant for the preparation of a medicament for the treatment of allergic and inflammatory respiratory conditions with cough, nasal congestion and the presence of mucus in the respiratory tract which comprises an effective amount of a non-sedating antihistamine in combination with an effective amount of an expectorant, and an effective amount of an nasal decongestant.

24. The use of claim 20-24 wherein the decongestant is pseudoephedrine or a pharmaceutically acceptable salt thereof.

25. The use of any of the claims 20-24 wherein the liquid pharmaceutical composition is stable to microbial contamination for periods of at least 4 months, preferably up to 36 months storage at room temperature.

26. The use of any of the claims 20-24 wherein the liquid pharmaceutical composition is stable to physical and chemical degradation of the active ingredients for periods of at least 4 months, preferably up to 36 months storage at room temperature.

27. The use of any of the claims 20-24 wherein the liquid pharmaceutical composition is stable to microbial contamination and to physical and chemical degradation of the active ingredients for periods of at least 4 months, preferably up to 36 months storage at room temperature.
